(19) United States
(12) Patent Application Publication
(10) Pub. No.: US 2025/0209612 A1
(43) Pub. Date: Jun. 26, 2025

(54) METHOD FOR THE ELABORATION OF FUNCTIONAL MAGNETIC RESONANCE IMAGES

(71) Applicant: QUANTABRAIN SRL, Pisa (PI) (IT)

(72) Inventors: Elisa FERRARI, Pisa (PI) (IT); Alessandro CELLERINO, Calci (PI) (IT); Davide BACCIU, Pisa (PI) (IT); Alessandra RETICO, Pisa (PI) (IT)

(73) Assignee: QUANTABRAIN SRL, Pisa (PI) (IT)

(21) Appl. No.: 18/848,941

(22) PCT Filed: Mar. 21, 2023
(86) PCT No.: PCT/EP2023/057150

§ 371 Date: Sep. 20, 2024

(30) Foreign Application Priority Data
Mar. 24, 2022 (IT) 102022000005873

(51) Int. Cl. G06T 7/00; G01R 33/48; G01R 33/56; G01R 33/563; G06T 7/20

(52) U.S. Cl. CPC G06T 7/0012; G01R 33/4806; G01R 33/5608; G01R 33/56308; G06T 7/20; G06T 2207/10021; G06T 2207/10088; G06T 2207/20081; G06T 2207/20084; G06T 2207/30016

(57) ABSTRACT

A method of processing functional magnetic resonance images on an organ or anatomical part of an individual using a machine learning algorithm through a neural network architecture is provided. The method includes obtaining scan data of a three-dimensional functional magnetic resonance video, which is spatio-temporal data relevant to the organ or anatomical part, obtaining data of the optical flow of the three-dimensional video, simultaneously applying to the scan data and to the optical flow data the machine learning algorithm, and obtaining output information on the organ or anatomical part. The learning algorithm is trained using an adversarial learning, wherein in the training of the learning algorithm, a desired output variable is set and the scan data of the three-dimensional magnetic resonance video are reprocessed based on the desired output variable.

### TECHNICAL FIELD

The present invention refers to a method of processing functional magnetic resonance images, of an organ or anatomical part of an individual, in particular using a machine learning algorithm with a neural network architecture. Furthermore, the present invention relates to an image processing system or data processing apparatus comprising means for carrying out the steps of said method. In addition, the present invention relates to a computer program capable of carrying out the steps of said method and to a computer readable medium comprising a program for implementing said method.

### STATE OF THE ART

Magnetic resonance is a radiological technique which is based on the physics of magnetic fields and allows to visualize the inside of an individual's body without performing surgical operations or administering ionizing radiation, such as for example in computed tomography, which uses X-rays. During a magnetic resonance, the individual is inside a very strong magnetic field that energizes the atoms that make up the body and orients them according to the direction of the magnetic field. When the magnetic field is deactivated, the atoms return to their natural orientation, releasing the accumulated energy and emitting a signal. Thanks to sophisticated systems, it is possible to intercept this signal and transform it into magnetic resonance images (imaging).

Magnetic resonance is used in many fields of application, for example in the neurological, neurosurgical, traumatological, oncological, orthopedic, cardiological, gastroenterological, etc. fields. In fact, in addition to being a relatively safe investigation technique for the patient compared to other imaging methodologies, the information obtained from the images is of a different nature compared to that of the other imaging methods as it is possible to discriminate between tissues on the basis of their biochemical composition. Furthermore, it is possible to obtain images of body sections in three different planes (axial, coronal, sagittal). There are several applications of magnetic resonance imaging, such as diffusion magnetic resonance imaging and functional magnetic resonance imaging (fMRI). The latter is a biomedical imaging technique which consists in the use of magnetic resonance imaging to evaluate the functionality of an organ or system, in a complementary way to structural imaging.

Recently, functional magnetic resonance is finding use for the diagnosis and staging of neurodegenerative diseases, such as multiple sclerosis Alzheimer's or disease. For such applications, machine learning algorithms based on the use of neural networks are often used.

These techniques are advantageous as they are able to autonomously learn complex operations which allow to process an image and extract the desired information from it without the developer listing the details of the operations to be performed. The objective of “autonomously learning” is reached with the so called “training phase”. Before the training process, a neural network algorithm consists of a sequence of layers that perform different mathematical operations, such as convolutional, pooling, padding, etc. with random parameters, meaning that the value of these parameters (often referred to as “weights”) is not tuned to process correctly the input data in order to extract the desired information. During the “training phase” the algorithm is fed with pairs of input data and their corresponding desired output. The desired outputs are instances of a variable that the user believes can be extracted from the input data. For instance, one can train an algorithm to recognize the mood of a subject based on a picture containing their facial expression. The parameters or weights are incrementally adjusted during the training phase in order to minimize the error between the desired outputs and the ones actually produced by the algorithm. Different metrics can be used to define the errors, the most common ones being “mean absolute error” and “mean squared error”. It is noted that a skilled person in the art would be able to define different metrics for measuring the error that are suitable for the particular data and application.

The training process does not have a pre-defined end, the user can stop the training process when the error reached is satisfactorily low according to their requirements or based on the time and computational resources available.

Once an algorithm is trained, it can be applied to new input data for which the desired output information is not available. The user can expect the error on the obtained output to be similar to that measured during the training phase. This second type of usage of the algorithm is called the “inference phase”.

However, the analysis of magnetic resonance images using neural networks is often complicated by various technical aspects related to the way these images are generated. In fact, magnetic resonance is currently a non-quantitative technique, i.e. it does not directly measure specific tissue properties, but metrics that are related to these properties. These relationships can be arbitrarily complex and are influenced by a large number of technical variables related to the equipment and the modalities of acquisition. Several studies show that since neural networks autonomously adjust the parameters to process fMRI images and extract information from them, often the outputs of the algorithm depend erroneously on these technical variables, even if these variables have no causal relationship with the information to extract, i.e. the desired output. This phenomenon whereby neural networks learn to extract incorrect information, dependent on other variables, also occurs for biological variables such as the sex and age of the subjects. In the context of neural networks, these variables—both technical and biological—are commonly referred to as confounding factors or variables and the phenomenon is called confounding effect.

The confounding effect occurs because, due to the limitedness of the training set, undesired biases can occur that generate spurious associations between the confounding variable and the desired output variable, which are not present in a larger and well sampled dataset. For instance, if the objective of the algorithm is to distinguish fMRIs belonging to subjects with and without a certain disease, but in the training set 80% of the subjects with the disease were acquired using the same scanner, the algorithm will learn a pattern according to which data taken from that scanner are predicted as belonging to diseased subjects.

The confounding variables are often already known to a person skilled in the type of data to be analyzed, but can be identified also using the so-called Confounding Index as described in the document “Measuring the effects of confounders in medical supervised classification problems: the Confounding Index (CI)” by E. Ferrari, A. Retico, and D. Bacciu (Artificial intelligence in medicine 103 (2020): 101804).

Adding to this problem is the additional complexity due to the fact that the result of a functional magnetic resonance scan is a 3D video of an individual's organ, for example the brain. In other words, this scan combines both spatial and temporal information. Extracting information from data with both spatial and temporal dimensions is quite complicated because spatial and temporal patterns are usually treated differently. Indeed, it is desirable to find spatial patterns invariant under rigid rotations or translations, while in time there is a clearly privileged direction. Furthermore, the identification of a spatial pattern could strongly depend on its temporal duration. For example, a pattern concerning a (figuratively speaking) moving object has a larger spatial dimension than that of the object itself, since the covered distance must also be considered.

Therefore, the objective of the present invention is to provide a method for processing functional magnetic resonance images which partially or totally solves the problems of the prior art. Specifically, the objective of the present invention is to provide a strategy for processing functional magnetic resonance images, with the aim of defining a protocol that allows taking into account the difference in the type of information carried by the spatial and temporal dimensions of the data. Also, an objective of the present invention is to reduce (i.e., minimize) the dependency of the learned pattern and consequently of the actual final output from a list of variables, for example set by the user. This list of variables set by the user can be defined as “user-defined confounding variables”.

### DESCRIPTION OF THE INVENTION

The aforementioned objectives are achieved by a method, by a system, by a computer program and by a computer readable medium according to the claims at the end of the present description.

In one aspect of the invention, a method of processing functional magnetic resonance images of an organ or anatomical part of an individual using a machine learning algorithm via a neural network architecture is provided. The method comprises the steps of obtaining scan data of a three-dimensional functional magnetic resonance video providing information on said organ or anatomical part, wherein the information is spatio-temporal data, obtaining data of the optical flow of said three-dimensional video, simultaneously applying to the scan data and to the optical flow data the machine learning algorithm, and obtaining output information on the organ or anatomical part, wherein said learning algorithm is trained using an adversarial learning and wherein in the training of the learning algorithm a desired output variable is set and the scan data of the three-dimensional magnetic resonance video are reprocessed based on said desired output variable.

The neural network architecture is based on the simultaneous analysis of the 3D video (4D fMRI) and its optical flow (OF), that can be either calculated internally by the algorithm or taken as input. Optical flow is a representation of video that enhances moving parts. The exact computation of the optical flow of a 3D video is an indeterminate problem. Thus, there exist different mathematical approximations to it, such as the Lucas-Kanade and the Horn-Schunck methods.

Thanks to the possibility of processing both the video scan data and the optical flow of the same, the learning of space-time aspects is improved.

According to an example, the information on the organ or anatomical part depends at least in part on a set of confounding variables and the dependency of the output information on said set of confounding variables is reduced, in particular minimized. In particular, the set of cofounding variables can be identified by a user together with the desired output variable.

Specifically, the adversarial learning allows the reduction, i.e. minimization, of the degree by which the learned pattern depends on the confounding variables, i.e. user-defined confounding variables.

In general, adversarial learning can be defined as a method in which a set of two or more models learn together by pursuing competing goals, usually defined on single data instances. For instance, a method to detect spam emails based on adversarial learning is composed by two subnets: a “generator” that learns to generate spam emails and a “detector” that learns to distinguish the spam emails output by the generator from true emails. The training of the “generator” and “detector” with competing tasks makes it possible to develop a “detector” that is much more robust to spam emails, with respect to its counterpart developed without an adversarial learning approach.

It should be noted that the peculiar characteristics of functional magnetic resonance images would make the separate use of the two techniques ineffective in sequence (i.e. adversarial learning and the simultaneous processing of the video and its optical flow).

Assuming to proceed initially with the first technique, i.e. the simultaneous processing of the fMRI video and its optical flow, and then with the second technique, i.e. the adversarial model, the output data from the first technique would undergo a dimensionality reduction not driven by adversarial learning; thus, information independent from the confounding variables may have been completely or largely discarded. Consequently, the subsequent application of the second technique would lead to much lower performance than what is obtained with the architecture described here. Assuming instead to initially apply the second technique, i.e. adversarial learning on the 3D video, and then to simultaneously process the output data from the second technique and the fMRI optical flow, this approach would be ineffective for two reasons. The output data from the second technique has been processed and no longer contains the same information as the original video. This could make it difficult to identify spatio-temporal patterns, which was instead the objective of the first technique that simultaneously processed video and its optical flow. Furthermore, the information contained in the optical flow, not having been processed with adversarial training, continues to be dependent on the confounding variables and therefore the information coming out of the system composed of the succession of the second technique and the first technique has not been fully processed in order to minimize its dependence from the confounding variables, i.e. from the user-defined confounding variables.

An advantage of the combined and non-sequential use of the two techniques is that the use of the optical flow provides the adversarial component with greater possibilities to identify and reduce (i.e. minimize) the dependence of the output from the confounding factors, providing an alternative representation of the data. In particular, this representation makes it easier for the network to learn temporal patterns, as it highlights the motion of objects (in a figurative sense). In the case of fMRI, for example, the flow of oxygenated and deoxygenated blood in the brain is highlighted. Providing a neural network with this new representation, obtained with extremely non-linear and complex calculations, significantly shortens the training time of the network.

The use of four-dimensional (4D) data, i.e., spatio-temporal data, offers several advantages over alternatives commonly explored in research. Usually, in fact, there are two options.
(1) The neural network processes a vector of image features extracted through engineered algorithms.
(2) The 4D image is segmented into multiple parts of reduced dimensionality parts which are processed separately.

In the first case, a portion of information is arbitrarily selected which could be irreparably compromised by dependence on confounding variables and/or from which it is not possible to extract the information of interest anymore. In the second case, it is not possible to identify patterns across several sections.

In another aspect of the invention, there is provided an image processing system or data processing apparatus comprising means, in particular a processor, for carrying out the steps of the processing method described herein.

In a further aspect of the invention, a computer program (product) is provided capable of carrying out the steps of the processing method described herein.

In an additional aspect of the invention, there is provided a computer readable medium comprising a computer program for carrying out the method described herein.

These and other aspects of the present invention will become clearer considering the following description of some preferred embodiments of the invention that are described below.

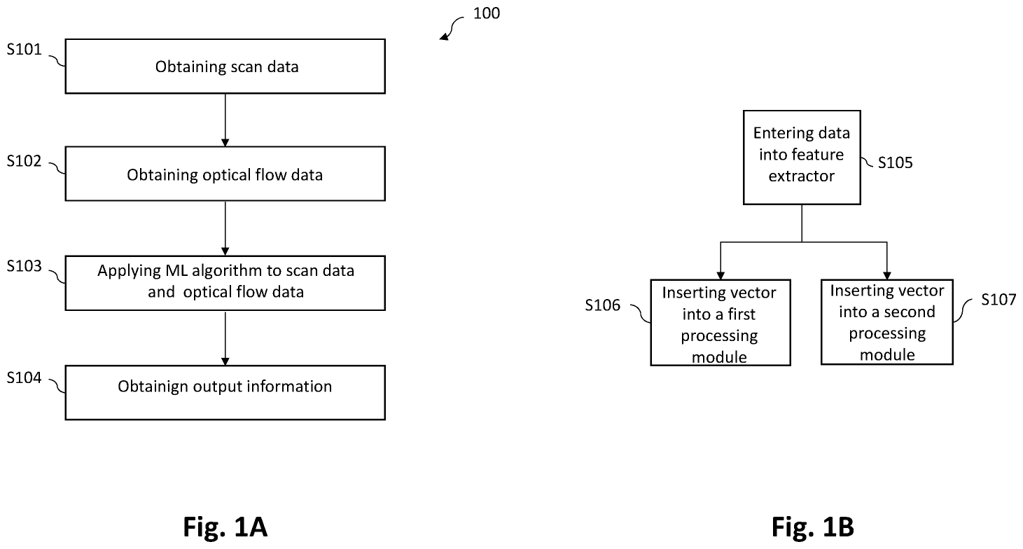

FIG. 1A-B show a flowchart of the method, according to an example.

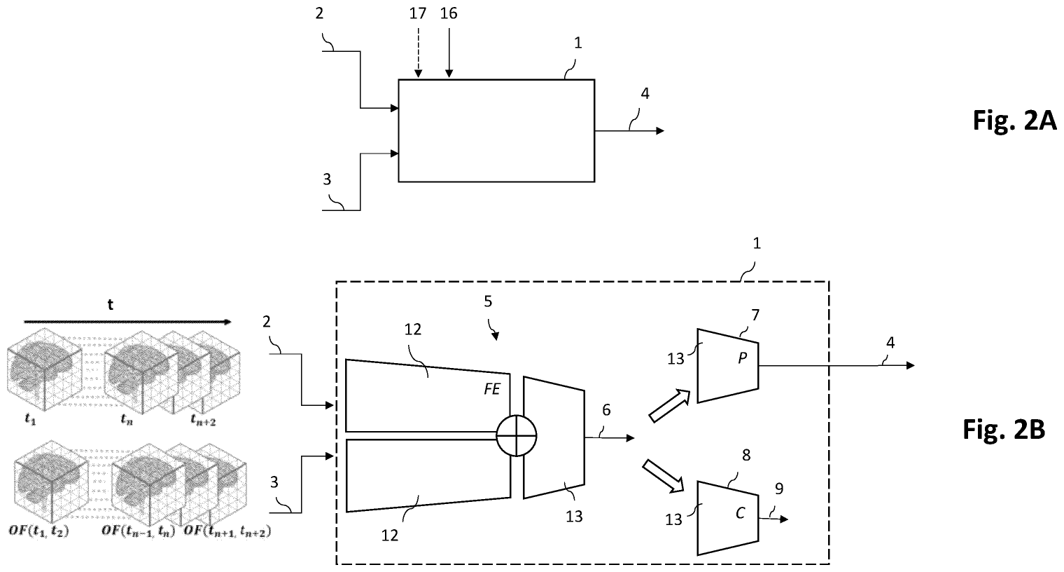

FIG. 2A-B show a schematic representation of a network architecture according to an example.

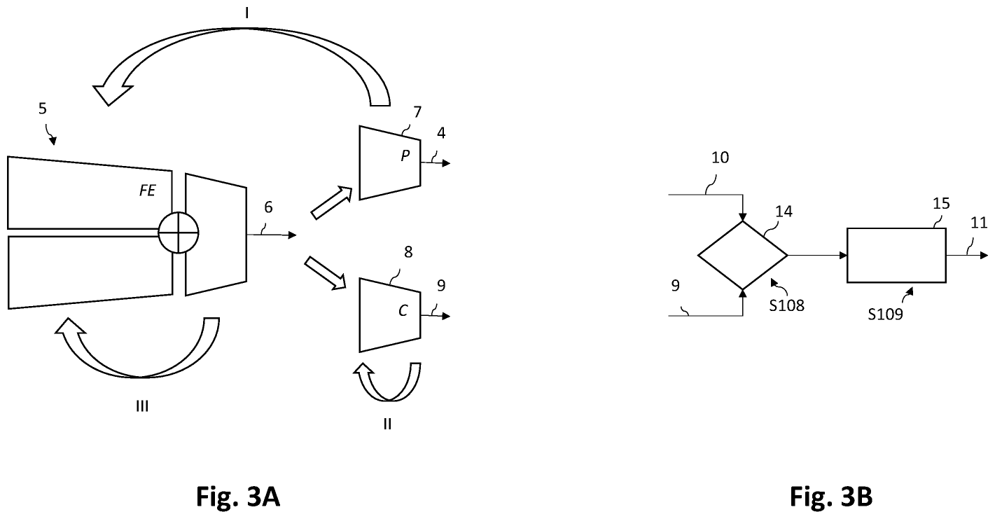

FIG. 3A-B show a schematic representation of the error back-propagation process applied to the network architecture as well as the phase of the correlation of the prediction vector according to an example.

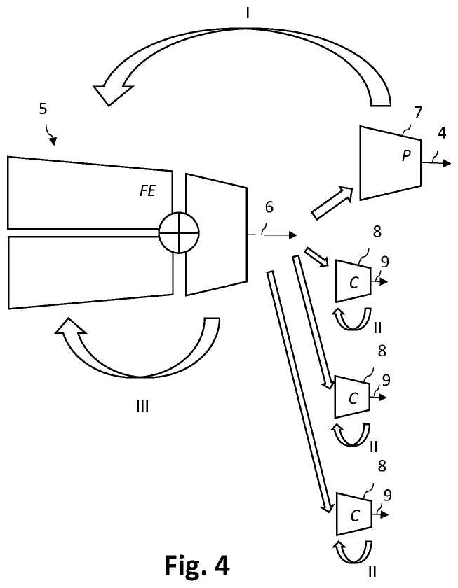

FIG. 4 shows a schematic representation of the error back-propagation process applied to the network architecture according to another example.

FIG. 1 shows a flowchart describing the functional magnetic resonance image processing method 100. The resonance images preferably refer to an organ or an anatomical part of an individual or patient. For example, images can be referred to the brain to study the flow of oxygenated and deoxygenated blood in the brain which provides an indirect measure of brain activity. The images can be referred to the heart to study how venous and arterial blood flows between the valves. The images can also refer to many other organs for which it is interesting to study not only their shape but their functionality such as lungs and kidneys.

These organs and other organs can be studied in humans or even animals.

Method 100 employs a machine learning algorithm based on a neural network architecture 1.

During the first step (or phase) S101 of method 100, scan data 2 of a functional magnetic resonance 3D video is obtained, which provides information about the patient's organ, such as the brain. This information is composed by spatio-temporal data. At step S102, data of an optical flow 3 of the 3D video is obtained. Optical flow is determined from the magnetic resonance video and can be represented by a tensor and describes a dense motion field with motion vectors at each pixel in a sequence of video frames. Subsequently, at step S103, the method 100 comprises the simultaneous application of the machine learning algorithm to both the scan data 2 and the optical flow data 3. Advantageously, the method 100 is based on an adversarial learning framework that is used to obtain (at step S104) output information 4 on the organ or anatomical part of the individual, for example in a way that minimizes the dependency of this information from the user-defined confounding variables. The output information 4 is for example a variable or an element that can be generated from the functional magnetic resonance image considering both the spatial and the temporal dimensions of the data regarding the patient's organ. The output information 4 can be also a reprocessed version of the original functional magnetic resonance image, in which this reprocessing takes into account the spatio-temporal characteristics of the original image. Together with the spatio-temporal characteristics of the original image, the reprocessing can take into account also the user-defined confounding variables.

During the “training phase”, a desired output variable 16 is set and the algorithm is fed with pairs composed by input data (scan data 2 and data of the optical flow their corresponding desired output variable 16, or the algorithm is fed with triplets composed by input data (scan data 2 and data of the optical flow 3), their corresponding desired output variable 16 and their corresponding user-defined confounding variables 17. The desired output variable 16 represents what a user believes can be extracted from the input data, i.e. the scan data 2. For example, the algorithm is trained to recognize a disease based on said scan data 2.

The user-defined confounding variables 17 are variables that affect the scan data 2 and whose influence on output information 4 is intended to be minimized by the user.

During the “training phase”, the scan data 2 of the three-dimensional magnetic resonance video are reprocessed based on the desired output variable 16 and on the user-defined confounding variables 17; in order to minimize the error (for example the mean absolute error, the mean squared error or other metrics) between the desired output variable 16 and the effective output information 4 produced by the algorithm and to minimize the dependency of the effective output information 4 from the user-defined confounding variables 17.

The reprocessing of the scan data 2 and the optical flow 3 is schematically illustrated in FIG. 2A in which both of them are inserted as an input (input) into the neural network architecture 1 for the application of the machine learning algorithm. The input data are paired with desired output variables 16. The input data can also be paired with user-defined confounding variables 17. Output information 4 is generated at the output (output).

By simultaneously processing both the video scan data 2 and the optical flow data 3 of said scan data 2, it is possible to improve the learning of space-time aspects.

According to an example, the information about the patient's organ that is composed by spatio-temporal data depends at least in part on a set of user-defined confounding variables 17. In particular, the method is trained to be resilient to these variables, using an adversarial learning framework, so that the dependency of the obtained output information 4 on said confounding variables 17 is reduced, in particular minimized. As mentioned above, the confounding variables 17 can be identified (set) by the user together with the desired output variable 16 and are associated to the input data, i.e. the scan data 2. However, the confounding variables 17 can also be identified (set) by the user in advance. During the training phase, the scan data 2, and its optical flow data 3, that depend on the confounding variables 17 are reprocessed based on the desired output variable 16. The (effective) output information 4 is extracted so as to minimize its dependency on the confounding variables 17. In other words, the algorithm is trained to eliminate, or strongly reduce, the influence of the confounding variables 17 on output information 4. Note that while the input data depend on confounding variables 17, the output data 4, thanks to the application of the algorithm, no longer depend (or depend in a reduced way) from the set of confounding variables 17.

As will be described in the following, the learning algorithm is trained using an adversarial learning and during the training a desired output variable 16 is set. The method comprises reprocessing the magnetic resonance video scan data 2 based on the desired output variable 16 to make the method resilient to the confounding variables 17. In other words, the trained method is applied to determine the output information 4 so that the scan data 2 and thus the images of functional magnetic resonance can be interpreted correctly, i.e. without them being influenced by confounding variables 17.

Assuming that from each fMRI we want to extract functional properties of the brain relating, for example, to the average, maximum and minimum speed of the blood and that the data available have been acquired by two different machines, one which presents artifacts on an important portion of the brain, there are two possible approaches in the literature to analyze the data.

A possible standard approach (1) to the problem consists in calculating image properties obtainable with engineered algorithms (such as the average grey levels per region, brain size, etc.) and then processing these data with a neural network to extract more complex information such as blood velocity. Another standard approach (2) is to use a network that receives 4D images and is trained to extract the desired speed properties.

In both situations (1) and (2), the calculation of these properties would be done on the whole brain and therefore the data acquired with the machine which presents significantly extended artifacts would have very different values that would not be comparable with those acquired with the other machine. So, the final extracted properties would depend on the acquisition machine.

By using method 100 described here instead, only a certain set of information is selected from the initial image which minimizes the ability to distinguish the data with respect to the “acquisition machine” confounding variable. Then, the velocity properties of the blood in the brain would be extracted on the basis of the artifact-free and comparable brain parts.

The output information 4 can be, for example, a categorical variable indicating whether the individual, on the basis of one or more functional magnetic resonance images, presents a certain pathology or not. However, the output information 4 can be any element that can be learned from the data, having any dimensionality. For example, this information can be a value, a vector, an image, a video, a tensor or even a graph.

Confounding variables 17 are well-known concepts in statistics and are variables that affect scan data 2 of the functional magnetic resonance three-dimensional video. In particular, a confounding variable can be any variable that has an influence on the values of the input data and that the user does not want to influence the output of the network.

In one example, the confounding variables 17 may include at least technical variables related to the equipment and techniques for acquiring the functional magnetic resonance image (such as scanner, acquisition parameters, acquisition modalities such as whether subjects have the eyes open or closed during the scan) and biological variables related to the characteristics of the organ or anatomical part analyzed (such as the patient's age or sex).

According to one example, the obtained scan data 2 refer to unprocessed functional magnetic resonance images. In particular, the obtained scan data 2 refer to functional magnetic resonance images that maintain their original size without any distortion.

In other words, the input functional magnetic resonance images can be unprocessed images, i.e. “raw”, i.e. without any modification, simply as they are produced by the scanner, or minimally processed, in the sense that minimal processing operations can be performed (noise reduction, skull stripping, intensity variations, etc . . . ) provided that the data remains 4D, i.e. of a spatio-temporal nature. Note in fact that, in order to be able to function adequately, the present method 100 uses information which comprises a temporal dimension in addition to the spatial one. Using unprocessed images reduces the complexity of the method. Furthermore, each pre-processing step introduces new noise and removes information from the original data, thereby increasing the probability that the pre-processed data does not contain any more information to be extracted or that the margin of error with which it can be extracted is greater.

Note that this method can also be advantageously used for the diagnosis of behavioral, neurodevelopmental or neurodegenerative disorders, such as Alzheimer's disease.

In fact, the diagnosis of various mental disorders/diseases is mainly based on the evaluation of behavioral traits that are difficult to measure, as a quantitative biomarker that characterizes them has not yet been found. Difficulty in diagnosis is accompanied by difficulty in treatment. In fact, due to the heterogeneity that characterizes these disorders, individuals with a mental disorder may require different care and treatments.

Since these disorders manifest themselves at the behavioral level, a complex biomarker (that is, one that can vary significantly from individual to individual) can be defined using artificial intelligence techniques on functional data that directly or indirectly measure brain activity at rest or during the execution of tasks. Such data must have both a spatial and a temporal component, such as functional magnetic resonance imaging (fMRI) and positron emission tomography (PET). The identification of a complex biomarker would also make it possible to stratify the population affected by a certain disease, in order to identify groups of individuals who probably respond in a similar way to treatments or cures. Thanks to the advantages offered by the present fMRI image processing method 100, the dependency of the output information 4 from the set of the confounding variables 17 (related to the acquisition equipment and/or to the sex and age of the subjects) is reduced, i.e. minimized. Furthermore, it is possible to distinguish the type of information carried by the spatial and temporal dimensions of the data. Therefore, in addition to processing fMRI images for their more correct interpretation, the present method 100 could also find application in the identification of a quantitative biomarker useful for the diagnosis and for the stratification of mental For example, thanks to the application of disorders. interpretability algorithms (or explaianbility) to the algorithm trained according to the present method, it is possible to identify, for each subject, the brain regions that are most significant for the diagnosis.

FIGS. 2A and 2B show in a block system the insertion of the scan data 2 and of the optical flow 3 inside the neural network architecture 1 and the output information 4 not dependent (or scarcely dependent) on the confounding variables 17. In particular, FIG. 2B shows, in detail, the structure of the neural network architecture 1 shown in FIG. 2A.

According to an example, simultaneously applying the machine learning algorithm to the scan data 2 and to the optical flow data 3 comprises entering said data into a features extractor 5 and extracting a reduced-size vector 6 (step S105 of FIG. 1B), wherein the features extractor 5 comprises a multi-channel, in particular two-channel structure.

The two-channel structure of the features extractor 5 serves to input and simultaneously process the scan data 2 and the optical flow data 3. As shown in FIG. 2B, the scan data 2 and the optical flow data 3 are each input in one of the two channels of the features extractor 5, respectively. The features extractor 5 (FE) serves to reduce the input information (fMRI+OF) to the reduced-size vector 6 which therefore contains a condensed representation of the input.

In an example, with reference to FIGS. 1B and 2B, the method 100 further comprises applying the machine learning algorithm to the reduced-size vector 6 and inserting said reduced-size vector 6 into a first processing module 7 to obtain output information 4 on the organ or anatomical part (step S106). The first processing module 7 can be a prediction module or Predictor (P) and uses the representation created by the features extractor 5 to generate the desired output, suitably processing the information.

Furthermore, the method 100 comprises applying the machine learning algorithm to the reduced-size vector 6 and inserting said reduced-size vector 6 into a second processing module 8 to obtain a prediction vector 9 on the confounding variables 17 associated to the organ information or anatomical part (step S107). The second processing module 8 can be a confounding variable prediction module or Confounder Predictor (C) and uses the representation created by the features extractor 5 to predict the factors confounding associated with each subject/individual: for example age, image acquisition modalities, etc. Confounding factors can be chosen by the user. However, in an alternative form of method 100, confounding factors can be automatically selected by a processor or electronic device.

As shown in FIG. 2B, the scan data 2 may comprise a set of fMRI images of an individual's brain as a function of time t. These data are entered into the features extractor 5 as they come out of the magnetic resonance scanning machine, i.e. without the need for specific pre-processing operations. However, all the images need to be of the same size, so spatio-temporal resampling may be necessary. The optical flow data 3 is the scan data 2 on which the optical flow has been calculated. There are several implementations of the optical flow calculation, the 100 method is compatible with all OF calculation methods. Optical flow data 3 has the same dimensionality as scan data 2.

The method 100 uses deep neural networks (also called deep learning) and is based on the succession of various mathematical operations called layers which produce increasingly abstract levels of representation of the input. In particular, both the two channels of the features extractor 5 comprise convolutional layers, pooling layers and drop-out layers 12. Furthermore, the features extractor 5 comprises a shared module having dense layers 13. Both the first processing module 7 and the second processing module 8 comprise dense layers 13.

Overall, the neural network is trained in an adversarial fashion. In particular, according to an example, the adversarial learning consists in favoring the learning of the first processing module 7 and opposing the learning of the second processing module 8. The weights of the network are updated during the training process through a back-propagation of the error, using 3 loss functions (I-III).

With reference to FIG. 3A, parameters associated with the features extractor 5 and the first processing module 7 are optimized to minimize the error of the first processing module 7, i.e., the error between the output information 4 and the desired output variable 16, pertaining to the input information, specified by the user. Furthermore, the parameters associated with the second processing module 8 are optimized to minimize the error of the second processing module 8, i.e., the error between the prediction vector 9 and the vector of the values of the user-defined confounding variables 17 pertaining to the input information. Finally, parameters associated with the features extractor 5 are optimized to maximize the error of the second processing module 8. The parameters of the network (number of layers, type of layer, number of neurons in each layer, activation functions, etc.) are determined with a preliminary search (also called grid-search). In other words, different configurations are tested and the user usually chooses the one that allows to obtain the highest performance.

In an example of a training scheme, the weights are updated through a particular sequence (I to III). However, a different sequence is also conceivable.

Firstly (I), the weights of the features extractor 5 and the first processing module 7 are updated based on a loss function to minimize the error on the output information 4. Subsequently (II), the weights of the second processing module 8 are updated based on a loss function to minimize the error on the prediction vector 9. Finally (III), the weights of the features extractor 5 are updated based on a loss function to maximize the error on prediction vector 9. This function is weighted with a hyperparameter that indicates how independent we want the network output to be from confounding variables 17. Usually, this constitutes a compromise, i.e., the more you want to obtain the independence from the confounding variables 17, the greater the error on the final prediction. It should be noted that whenever we refer to the minimization or maximization of the error of a module, we mean the minimization or maximization of the error committed in the estimation of the vector or output information from that module.

The same error metrics or different metrics can be used to update the various modules. For example, a different metric can be used for the features extractor 5 update than for the second processing module 8 update. Some metrics that can be used are cross-entropy for categorical variables, mean square error or mean absolute error for continuous variables. If the information to be extracted is a vector containing both categorical and continuous variables, the correlation between the predicted vectors and the vector containing the desired outputs can be used.

In one example, the confounding variables 17 are represented by a vector of confounding variables 10. Referring to FIG. 3B, the method 100 further comprises performing a correlation between the vector of confounding variables 10 and the prediction vector 9 (step S108) and measuring a correlation value 11 at the end of the training of the neural network, wherein the output information 4 on the organ or anatomical part depends on the vector of confounding variables 10 in proportion to said correlation value 11 (step S109).

The prediction vector 9 can be correlated to the vector of confounding variables 10 in a correlation module 14 and the measurement of the correlation value 11 can take place in a measurement module 15. It should be noted that this correlation value 11 is an index of the goodness of applying machine learning algorithm on fMRI images.

If measurement module 15 uses Pearson's correlation as a metric, then the index assessing how dependent output 4 is on the confounding variables 17 can be said to be between 0 and 1. Where 0 indicates no dependence and 1 indicates perfect proportionality. Other indexes are possible with different ranges.

In other words, this value indicates to what extent the output information 4 depends or not on the confounding variables 17. For example, fMRI images of a patient's brain can be used to determine whether or not (and to what degree) the patient is affected by a certain disease, such as a neurodegenerative disease. In the event that fMRI images were analyzed without applying the method described here, the information would be influenced by confounding variables 17, such as the type of machine used to acquire the images. By instead applying the present method 100, it is possible to obtain more correct information, reducing the errors due to the presence of confounding variables 17 (for example the type of machine used).

FIG. 4 describes a representation of the adversarial learning process applied to the network architecture according to an alternative approach compared to that of FIG. 3A. Differently from the configuration of FIG. 3A, the reduced-size vector 6 can be inserted into more than one second processing module 8 (in the figure are shown three second processing modules 8 in parallel), each of them dedicated to output a subset of the user-defined confounding variables 17 associated to the organ information or anatomical part.

The parameters associated with the features extractor 5 and the first processing module 7 are optimized to minimize the error of the first processing module 7, i.e., the error between the output information 4 and the desired output variable 16, pertaining to the input information, specified by the user. Furthermore, the parameters associated with each of the second processing module 8 are optimized to minimize the error of each second processing module 8, i.e., the error between each prediction vector 9 and the vector of the values of each subset of the user-defined confounding variables 17 pertaining to the input information. In other words, each second processing module 8 is configured to learn one single confounding variable or a group of confounding variables so that the total amount of confounding variables are not learned by a single second processing module 8, as in FIG. 3A but is distributed among a plurality of second processing modules 8. Finally, the parameters associated with the features extractor 5 are optimized to maximize the error of each second processing module 8.

As regards the optimization of the weights, firstly (I), the weights of the features extractor 5 and the first processing module 7 are updated based on a loss function to minimize the error on the output information 4. Subsequently (II), the weights of each second processing module 8 are updated based on one or more losses functions to minimize the error of each second processing module 8. Finally (III), the weights of the features extractor 5 are updated based on a loss function to maximize the errors on the different prediction vectors 9. In this alternative representation of the adversarial learning process, it is possible to define a loss that prioritizes the maximization of the error of one or more of the second processing modules 8 over the others. It is also possible to develop each second processing module 8 with a different number of internal parameters. The more internal parameters are present in a second processing module, the more complex the information contained in the prediction vector 9 of that module can be. The measurement of a correlation value as shown in FIG. 3B applies also to the representation of FIG. 4. However, the measurement should be intended considering the correlation step for each of the plurality of second processing modules 8.

The method can be applied to any image with the same dimensions of the images used to train the algorithm, irrespectively of the organ or anatomical part depicted in it, however if the algorithm was trained on images depicting different organs or anatomical parts, the output information 4 will unlikely be the desired output information 16.

In general, the method (100) can be successfully applied only to images of the organ or anatomical part for which the algorithm was previously trained. Also, the choice of the desired output variable 16 and of the confounding variables 17 should be made by the user according to the organ or anatomical part that is intended to be analyzed. However, the method can be always trained on any organ or anatomical part.

In one example, the method (100) may be successfully applied to any organ or anatomical part, if one of the confounding variables 17 defined by the user is the organ/anatomical part variable. For instance, it may be possible to train the algorithm to identify a property in the image such as average water density depicted in the video and make the extraction of this information independent from the organ depicted in the image, by setting the organ variable as one of the user-defined confounding variables 17. In this case even if the algorithm was trained on brain and heart images, it may be successfully applied to lung and kidney images.

In another example, the method (100) may be successfully applied to any organ or anatomical part if the output variable can take an undefined value. For instance, it may be possible to train the algorithm to classify the organ or anatomical part depicted in the image. If the algorithm is trained to output “a” for images that depict a heart, “b” for images that depict a brain and “c” for images that depict any other organ or anatomical part (the said undefined value).

In this case, even if the training set contains data depicting only hearts, brains, lungs and kidneys, the method may successfully output “c” when applied to any other organ.

To the method described above, a person skilled in the art, in order to satisfy further and contingent needs, may make numerous further modifications and variations, all however included in the scope of protection of the present invention as defined by the attached claims.

1. (canceled)

2. (canceled)

3. The method according to claim 2, wherein the set of confounding variables is identified by a user together with the desired output variable.

4. (canceled)

5. (canceled)

6. The method according to claim 5, wherein the confounding variables are defined by a vector of confounding variables and wherein the method further comprises:
performing a correlation between the vector of confounding variables and the prediction vector; and
measuring a correlation value at the end of the training of the neural network, wherein the output information on the organ or anatomical part depends on the vector of confounding variables in proportion to said correlation value.

7. The method according to one claim 15, wherein:
a. the confounding variables are variables that affect the scan data of the functional magnetic resonance three-dimensional video; and/or
b. the confounding variables include at least technical variables related to the equipment and techniques for acquiring the functional magnetic resonance image and biological variables related to the characteristics of the organ or anatomical part analyzed.

8. The method according to claim 15, wherein:
a. the obtained scan data refer to unprocessed functional magnetic resonance images; and/or
b. the obtained scan data refer to functional magnetic resonance images that maintain their original size without any distortion.

9. (canceled)

10. The method according to claim 15, wherein:
a. parameters associated with the features extractor and the first processing module are optimized to minimize the error of the first processing module; and/or
b. parameters associated with the second processing module are optimized to minimize the error of the second processing module; and/or
c. parameters associated with the features extractor are optimized to minimize the error of the first processing module and maximize the error of the second processing module.

11. An image processing system or data processing apparatus comprising means, in particular a processor, for carrying out the steps of the method according to claim 15.

12. (canceled)

13. (canceled)

14. (canceled)

15. A method of processing functional magnetic resonance images of an organ or anatomical part of an individual using a machine learning algorithm through a neural network architecture, wherein the method comprises:
training a machine learning algorithm to extract a desired output variable from functional magnetic resonance images of an organ or anatomical part of individuals, that depend at least in part on a set of confounding variables, using an adversarial learning method, in which the dependency of the output information on said set of confounding variables is progressively reduced during training; the machine learning algorithm comprises:
the calculation of the optical flow of the training images;
a multi-channel feature extractor module that simultaneously processes the functional magnetic resonance images and their optical flow and extracts a reduced-size vector;
a first processing module that processes the reduced-size vector and predicts the output variable;
a second processing module that processes the reduced-size vector and predicts the confounding variables; and
the training of the machine learning algorithm is favoring the learning of the first processing module and opposing the learning of the second processing module;
obtaining scan data of a three-dimensional functional magnetic resonance video, which provide information on said organ or anatomical part, wherein the information is a spatio-temporal data, that depends at least in part on the set of confounding variables;
obtaining data of the optical flow of said three-dimensional video;
simultaneously applying the machine learning algorithm to the scan data and to the optical flow data; and
obtaining output information on the organ or anatomical part based on the application of the machine learning algorithm to the scan data and the optical flow data; and
getting the output information, wherein the dependency of the output information on said set of confounding variables is reduced.

16. A method of processing brain functional magnetic resonance images of an individual using a machine learning algorithm through a neural network architecture in order to diagnose a behavioral, neurodevelopmental, or neurodegenerative disorder, wherein the method comprises:
training a machine learning algorithm using an adversarial learning, wherein in the training of the learning algorithm a desired output variable (that is useful to diagnose a behavioral, neurodevelopmental, or neurodegenerative disorder) is set and the scan data of the three-dimensional brain magnetic resonance video are reprocessed based on said desired output variable;
obtaining scan data of a three-dimensional brain functional magnetic resonance video wherein the information is a spatio-temporal data;
obtaining data of the optical flow of said three-dimensional video;
simultaneously applying to the scan data and to the optical flow data the trained machine learning algorithm; and
obtaining output information on brain based on the application of the machine learning algorithm to the scan data and the optical flow data; and diagnosing one of a behavioral disorder, a neurodevelopmental disorder, or neurodegenerative disorder based on the output information.
